(19) United States
(12) Patent Application Publication
(10) Pub. No.: US 2012/0065445 A1
(43) Pub. Date: Mar. 15, 2012

(54) Process For Synthesis of 9,9'-Dianthracene

(76) Inventors: Lifei Cai, Beijing (CN); Lei Dai, Beijing (CN); Hongyu Zhao, Beijing (CN)

(21) Appl. No.: 13/321,799

(22) PCT Filed: May 5, 2010
(86) PCT No.: PCT/CN2010/072441

§ 371 Date: Nov. 21, 2011

(30) Foreign Application Priority Data
May 22, 2009 (CN) 200910085439.8

(51) Int. Cl. C07C 1/20

(52) U.S. Cl. 585/469

(57) ABSTRACT

This invention, which involves “the synthetic method of 9, 9′-biantnthracine”, belongs to the field of synthetic technology of organic electroluminesent materials. Synthetic method of 9, 9′-bianthracine is to add anthraquinone as raw material and zinc as reducing agent in glacial acetic acid solution, then batch addition of hydrochloric acid at 70-120°, maintain the temperature unchanged and react, then 9, 9′-bianthracine is achieved. This invention uses one-step method to synthesize 9, 9′-bianthracine, which reduces not only cost but also generation of side products, in addition, the products obtained need no purification and can be directly used to synthesize related similar compounds, therefore, it is very suitable for large-scale industrial production.

### TECHNICAL FIELD

This invention belongs to the field that related to organic electroluminesent materials synthetic technology, and particularly involves the synthetic method of 9, 9′-bianthracine which serves as intermediate of field-effect transistor materials and organic electroluminesent materials.

### TECHNICAL BACKGROUND

In 1987, Ching W. Tang of Kodak Company in the US successfully worked out sandwich-type double-layer light-emitting devices by using Alq₃ as luminescent layer and aromatic diamines as hole transport layer (Tang C. W.,et al. Applied Physics Letters, 1987, 51, 913). In 1990, Burroughes J. H. et al. of Cambridge University developed organic polymer light-emitting diodes (Burroughes J. H. et al. Nature, 1990, 347, 5395). These major breakthroughs pushed development of organic light emitting technology significantly. OLED products have already been commercialized, its advantages, such as soft colors and high definition, are attracting the attention of an increasing number of people. And its fatal weakness of lifespan and stability can be improved through looking for new light-emitting materials and reforming manufacturing technology. As a result, synthesis and performance of new organic light-emitting materials are the focus of current research. Scientists of various countries have devoted much energy to research and development of this technology, consequently, an increasing number of organic electroluminescent materials have been developed and applied, and among which anthraquinone compounds are a kind of organic light-emitting materials with special luminescence properties and good performance. The band-gap of 9, 9′-bianthracine crystal is about 3 eV, only light with wavelength below 410 nm can stimulate it, so it is very stable in the air. In addition, hole mobility of crystal based on bianthracine at room temperature can reach 3 cm²/V·s therefore, bianthracine field effect transistor has attracted a considerable amount of interests of research recently. Meanwhile, its derivative is a kind of very promising blue light-emitting material (M. H. Ho, Y. S. Wu, S. W. Wen, et al., Appl. Phys. Lett., 2006, 89, 252903/1-3.), and very important to study and manufacture of blue, white organic electroluminescent devices.

Anthraquinone compounds, which is the ADN, is a kind of very good blue light-emitting material, and its energy level is 3.1 ev (J.-H. Jou, Ch.-P. Wang, et al., Organic Electronics, 2007, 8, 29-36.). Anthraquinone compounds can be synthesized through bianthracine , but the current synthetic method of bianthracine and its dibromide has problems such as low yield and complex after treatment (Yuliang Mai, Guangdong Chemical Industry, 2007, 34, 9; J. Chem. Soc. 1949,267-269.). It is known from literature that bianthracine was mainly synthesized through anthrone, the yield was 40-50% (J. Chem. Soc., 1949, 267-269) which is not suitable for industrialization, through Yuliang Mai improved the synthesis, phosphorus pentoxide was easy to wrap raw materials and many raw materials did not participate in the reaction. Overall, there are only three synthetic methods of bianthracine at present: (1) in most literatures, anthrone is adopted as raw material, hydrochloric acid and acetic acid as solvent, stannum reduction, and yield is around 50%. (2) anthraquinone is used as raw material, hydrochloric acid and acetic acid as solvent, stannum reduction, and yield is around 50%. (3) Use 9-bromoanthracene as raw material, grignard reaction, oxidative coupling of copper chloride, the yield is 55%. It can be seen that yields of these three methods are all not high and after treatment is very complex, therefore, they are not suitable for industrialization. Though Yuliang Mai improved the anthrone method and separated coupling and closed loop, phosphorus pentoxide was easy to wrap raw materials and half of raw materials did not participate in the reaction, as a result, this method is not desirable as well.

In light of the current problems existed in bianthracine synthesis, new method that suitable for industrialization needs to be explored.

### CONTENT OF THIS INVENTION

Aiming at defects in the above field, this invention improves production process by synthesizing 9, 9′-bianthracine in one step, which not only reduces cost, but also conducive to industrial production.

Synthetic method of 9, 9′ -bianthracine: add anthraquinone as raw material and zinc as reducing agent in glacial acetic acid solution, then batch addition of hydrochloric acid at 70-120°, maintain the temperature unchanged and react, then 9, 9′-bianthracine is achieved.

It is stated that the reaction should last 2-15 hours at 70-120°.

It is optimal to react 2-10 hours at 80-110°.

The stated reaction should take place under protection of nitrogen.

The stated volumetric proportion of glacial acetic acid and hydrochloric acid is 4:1.

End of the stated reaction is detected by TLC method.

The stated synthetic method also includes after treatment procedures which consists of cooling reaction solution, filtrating, washing product with solvents.

The stated solvents are toluene, xylene, ethanol, methanol and/or isopropyl alcohol.

During reduction process of anthrone, the side product of anthrapinacolin is very easy to be produced, the main reason is that diol obtained from reduction rearranges under the environment of acid, which cuts down yield. Based on repeated experimental results of literatures, we adjust proportion of hydrochloric acid and acetic acid, as well as rate of adding raw materials, and by using anthraquinone as raw material, zinc as reduction agent and synthesizing bianthracine in one step, costs are lowered, therefore, this method is suitable for industrialization. It can be seen from the result of detection that the method we adopted does not involve cyclic ether, open loop and rearrangement product (anthrapinacolin) mentioned in literature, but only a small amount of intermediate. Electron transfer quickly occurred to zinc powder we chose under the environment of hydrochloric acid, and the zinc chloride produced is a kind of mild catalyst, thus reducing generation of side product (anthrapinacolin).

During after treatment, since products will be separated out after cooling, so if high yield and products of high purity are wanted, cleaning solvent that can clean reaction solvent, unreacted raw materials and impurities is in need, meanwhile, products cannot dissolve too much in cleaning solvent, therefore, it is necessary to select cleaning solvent carefully. Cleaning solvent selected in this invention includes toluene, xylene, ethanol, methanol and/or isopropyl alcohol, and the optimal choice is toluene.

The compound designed in this patent can be synthesized in accordance with the following process:

(1) Add acetic acid, zinc powder, anthraquinone in reaction flask, stir and fill it with nitrogen, and then drop hydrochloric acid in it while maintaining the temperature at 70°-120°.

(2) After addition of hydrochloric acid, maintain the temperature at 70° -120° and react 2-15 hours, then cool it, separate solids out, filtrate the solids and purify them.

This invention uses one-step method to synthesize 9, 9′-bianthracine, which reduces not only cost but also generation of side products, in addition, the products obtained can be directly used to synthesize other similar compounds, therefore, it is very conducive to industrial production.

### PRACTICAL IMPLEMENTATION MODES

### Implementation Example 1:

Add 600 ml glacial acetic acid, 25 g anthraquinone, and 55 g zinc powder in boiling flask-4-neck, fill it with nitrogen, heat and stir it. Drop 150 ml hydrochloric acid slowly while maintaining the temperature between 80° to 90°. After that, react at 90°, the color of the mixture is growing deep and solids are separated out gradually. After 8-hour reaction, no raw materials are left on tap, then stop the reaction, filtrate the solids and purify them with toluene, after drying them, 17 g products are gained with yield of 80%.
m.p.>300°;
1HNMR(CDCl3): 7.00˜7.19 (m , 8 H), 7.42˜7.48 (m , 4 H), 8.27 (d, J=12, 3 Hz, 4 H), 8.67(s, 2 H); ESIMS z/e: 355.1[M+H]+

1. A method of synthesizing 9, 9′-bianthracine comprising: adding anthraquinone as raw material and zinc as reducing agent in glacial acetic acid solution, then batch addition of hydrochloric acid at 70-120°, maintain the temperature unchanged and react, then 9, 9′-bianthracine is achieved.

2. The method according to claim 1, wherein the reaction should last 2-15 hours at 70-120° after addition of hydrochloric acid.

3. The method according to claim 2, wherein the stated temperature is 80°-110° when hydrochloric acid is added, and then the reaction should last 2-10 hours at 80°-110° .

4. The method according to claim 1, wherein the stated reaction should take place under protection of nitrogen.

5. The method according to claim 1, wherein the stated volumetric proportion of glacial acetic acid and hydrochloric acid is 4:1.

6. The method according to claim 1, wherein the end of the stated reaction is detected by TLC method.

7. The method according to claim 1, wherein the stated synthetic method also includes after treatment procedures which consists of cooling reaction solution, filtrating, washing product with solvents.

8. The method according to claim 7, wherein the stated solvents are toluene, xylene, ethanol, methanol and/or isopropyl alcohol.
